(19) United States
(12) Patent Application Publication
(10) Pub. No.: US 2022/0382480 A1
(43) Pub. Date: Dec. 1, 2022

(54) METHOD, SYSTEM, APPARATUS FOR DATA STORAGE, DECODING METHOD, AND STORAGE MEDIUM

(71) Applicant: Guangzhou University, Guangzhou (CN)

(72) Inventors: Xu YANG, Guangzhou (CN); Xiaolong SHI, Guangzhou (CN); Xiaoli QIANG, Guangzhou (CN)

(21) Appl. No.: 17/469,048

(22) Filed: Sep. 8, 2021

(30) Foreign Application Priority Data
May 27, 2021 (CN) 202110583430.0

(51) Int. Cl. G06F 3/06; G06F 7/58; G16B 50/30

(52) U.S. Cl. CPC G06F 3/0655; G06F 7/588; G06F 3/0604; G06F 3/0679; G16B 50/30; H03K 19/21

(57) ABSTRACT

The invention discloses a method, a system, an apparatus for data storage, and a storage medium. The method for data storage medium includes: acquiring first data; grouping the first data to obtain K packet sub-data; inputting a preset primer into a random generator to obtain 4T random number sequences, 4^{T}>K; determining the packet sub-data corresponding to the ith random number sequence, and performing exclusive or (XOR) operation on the determined packet sub-data to obtain data information DATAi, and obtaining a DNA molecular chain according to the data information DATAi, the preset primer and the generation times capacity of the random generator; performing DNA sequence synthesis on the plurality of DNA molecular chains to obtain target storage data. In the disclosure, in the process of coding the first data to obtain a DNA molecular chain, a random generator is added to greatly simplify the coding process and implement efficient and accurate coding on the first data. The disclosure may be widely applied to a field of data storage technologies.

### TECHNICAL FIELD

The disclosure relates to a field of data storage technologies, and particularly to a method, a system and an apparatus for data storage, and a storage medium.

### BACKGROUND

With the development of science and technology, data is increasing rapidly. It is an important problem how to store massive data. In order to solve this problem, related researches on data storage by using deoxyribonucleic acid (DNA) have emerged. All information is stored in the form of a DNA chain, which theoretically enables information to be stored for a longer period of time without any loss of data. With regard to the current DNA storage technology, when data at a specific location needs to be acquired, all the data stored in the DNA needs to be read and screened, and there is no way to read only a portion of data oriented to a specific location, with low efficiency and defects.

### SUMMARY

The present disclosure is intended to solve one of technical problems in the related art to at least certain extent.

Therefore, one purpose of the embodiments of the disclosure is to provide a method, a system, an apparatus for data storage, a decoding method, and a storage medium.

To achieve the above technical purpose, the technical solutions in the embodiments of the disclosure include:

In a first aspect, the embodiments of the disclosure provide a method for data storage. The method includes:

acquiring first data;

grouping the first data to obtain K packet sub-data, the K being a positive integer;

inputting a preset primer into a random generator to obtain 4^{T} random number sequences, T being a generation times capacity of the random generator, and 4^{T}>K, a preset ratio of the content of guanine and cytosine in the preset primer prefix to the total content of guanine, cytosine, adenine and thymine contained in the preset primer;

determining the packet sub-data corresponding to the ith random number sequence, and performing exclusive or (XOR) operation on the determined packet sub-data to obtain data information DATAi, i being a natural number and 1≤i≤4^{T}, and obtaining a DNA molecular chain according to the data information DATAi, the preset primer and the generation times capacity of the random generator;

performing DNA sequence synthesis on the plurality of DNA molecular chains to obtain target storage data.

Further, grouping the first data to obtain K packet sub-data, includes:

determining a data length and a packet length of the first data;

obtaining K packet sub-data according to the data length and the packet length.

Further, inputting a preset primer into a random generator to obtain 4^{T} random number sequences, is specifically:

controlling cycle number j, outputting a random integer in a range [0, 2K] according to the input preset primer by the random generator, and converting the random integer sequence into a random number sequence DATAj in a binary form;

1≤j≤4^{T}.

Further, each of the random number sequences includes K random bits; determining the packet sub-data corresponding to the ith random number sequence, and performing XOR operation on the determined packet sub-data to obtain data information DATAi, includes:

when judging that the value of the mth random bit of the ith random number sequence is 1, selecting the packet sub-data corresponding to m random bits, m being an integer and 1≤m≤K;

performing XOR operation on the selected packet sub-data to obtain the data information DATAi.

Further, the storage method further includes randomization of the DNA molecular chain. The method includes:

inputting a preset primer into a random generator to obtain a random integer sequence;

converting the random integer sequence into a binary sequence or a corresponding base sequence, generating a degree distribution sequence under the guidance of the generation times of the random generator, and guiding the data information to perform XOR operation.

In a second aspect, the embodiments of the disclosure provide a decoding method. The method includes:

decoding the target storage data.

In a third aspect, the embodiments of the disclosure provide a system for data storage.

The system includes:

a data acquiring module, configured to acquire first data;

a packet module, configured to group the first data to obtain K packet sub-data, the K being a positive integer;

a random number sequence acquiring module, configured to input a preset primer into a random generator to obtain 4^{T} random number sequences, T being a generation times capacity of the random generator, and 4^{T}>K, a preset ratio of the content of guanine and cytosine in the preset primer prefix to the total content of guanine, cytosine, adenine and thymine contained in the preset primer;

a packet determining module, configured to determine the packet sub-data corresponding to the ith random number sequence, and perform exclusive or (XOR) operation on the determined packet sub-data to obtain data information DATAi, i being a natural number and 1≤i≤4^{T}, and obtain a DNA molecular chain according to the data information DATAi, the preset primer and the generation times capacity of the random generator;

a synthesis module, configured to perform DNA sequence synthesis on the plurality of DNA molecular chains to obtain target storage data.

Further, each of the random number sequences includes K random bits. The packet determining module includes:

a judging unit, configured to, when judging that the value of the mth random bit of the ith random number sequence is 1, select the packet sub-data corresponding to m random bits, m being an integer and 1≤m≤K;

an XOR operation unit, configured to perform XOR operation on the selected packet sub-data to obtain the data information DATAi.

In a fourth aspect, the embodiments of the disclosure provide an apparatus for data storage. The apparatus includes:

at least one processor; and

at least one memory configured to store at least one program;

when the at least one program is executed by the at least one processor, the at least one processor is caused to implement the method for data storage.

In a fifth aspect, the embodiments of the disclosure provide a storage medium stored with programs executable by a processor, the programs executable by the processor being configured to implement the method for data storage when executed by the processor.

Advantages and beneficial effects of the present disclosure will be set forth in part in the following description, and in part will become obvious from the following description, or may be learned by practice of the disclosure.

In embodiments of the disclosure, in the process of coding the first data to obtain a DNA molecular chain, a random generator is added to greatly simplify the coding process and implement efficient and accurate coding on the first data, and a primer of a DNA molecular chain is configured as a seed of a random generator to maximize the function of the primer.

### BRIEF DESCRIPTION OF THE DRAWINGS

In order to explain the technical solutions in embodiments of the present disclosure or the related art more clearly, the drawings described in the embodiments or the related art will be briefly introduced below. It should be understood that the drawings described as below are only some embodiments of the present disclosure. Those skilled in the art may obtain other drawings from these drawings without creative work.

FIG. 1 is a flow diagram of a specific embodiment of a method for data storage in the disclosure;

FIG. 2 is a diagram of a specific embodiment of a structure of a system for data storage in the disclosure;

FIG. 3 is a diagram of a specific embodiment of a structure of an apparatus for data storage in the disclosure.

FIG. 4 is a diagram of one embodiment showing a data structure related to the current disclosure.

### DETAILED DESCRIPTION

Embodiments of the present disclosure are described in detail below, and examples of embodiments are illustrated in the accompanying drawings, in which the same or similar labels represent the same or similar elements or elements with the same or similar functions. The embodiments described below with reference to the drawings are exemplary, are only configured to explain the present disclosure and are not to be construed as a limitation of the present disclosure. The block numbers in the following embodiments are set only for illustration, the sequence between blocks is not limited, and the execution sequence of the blocks in the embodiments may be adaptively adjusted according to the understanding of those skilled in the art.

For a method and a system for data storage according to embodiments of the present disclosure with reference to the drawings below, a method for data storage according to the embodiment of the present disclosure is described first with reference to the accompanying drawings.

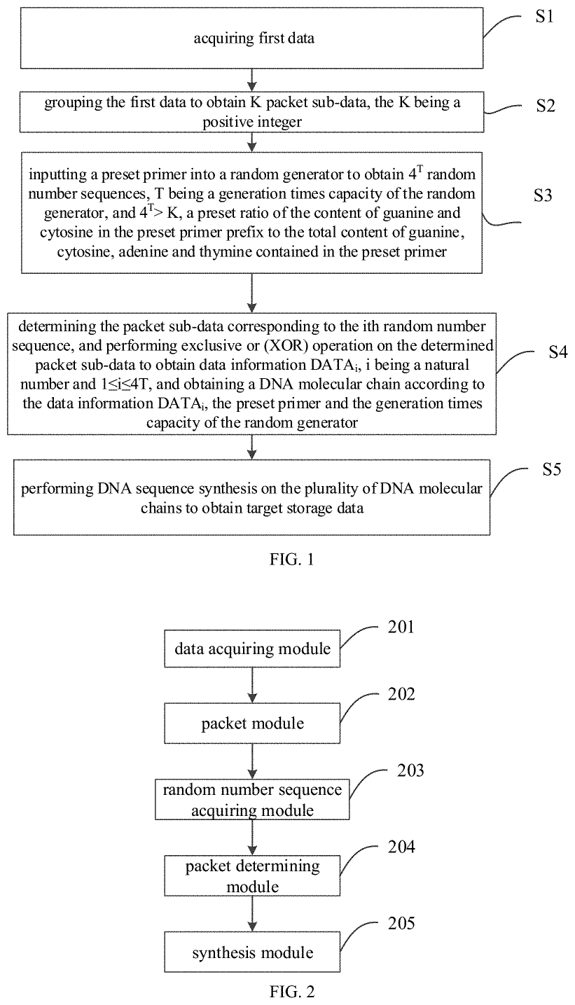

Referring to FIG. 1, the method for data storage described in embodiments of the disclosure includes:

at S1, first data is acquired;

at S2, the first data is grouped to obtain K packet sub-data, the K being a positive integer;

at S3, a preset primer is input into a random generator to obtain 4^{T} random number sequences, T being a generation times capacity of the random generator, and 4^{T}>K, a preset ratio of the content of guanine and cytosine in the preset primer prefix to the total content of guanine, cytosine, adenine and thymine contained in the preset primer;

at S4, the packet sub-data corresponding to the ith random number sequence is determined, and exclusive or (XOR) operation is performed on the determined packet sub-data to obtain data information DATAᵢ, i being a natural number and 1≤i≤4^{T}, and a DNA molecular chain is obtained according to the data information DATAᵢ, the preset primer and the generation times capacity of the random generator;

at S5, DNA sequence synthesis is performed on the plurality of DNA molecular chains to obtain target storage data.

Specifically, DNA storage is target information to be stored, that is, first data converted into the DNA base coding stored in a DNA chain, and when the data needs to be read, the DNA chain (sometimes PCR amplification is required on the DNA chain first and then sequencing operation is performed) is sequenced to obtain a corresponding base sequence, and the corresponding base sequence is changed into information that may be recognized by the electronic computer through a series of conversions for data recovery.

First, the first data is grouped to obtain K packet sub-data, that is, S₁, S₂, S₃ . . . Sₖ, the data length of each packet sub-data being fixed.

The preset primer is a DNA sequence specially designed for subsequent PCR amplification or sequencing with a specific base arrangement structure, which is predetermined and recorded before coding the first data.

The preset primer is input to a random generator as a seed of a random generator, to obtain a plurality of random numbers. The generation times capacity of the random generator is T, 4^{T} is the generation times of the random generator, and the random generator may generate 4^{T} random numbers by controlling the cycle number of the random generator.

For example, the data length of each packet sub-data is S=4200 (bit), N=40 (nt), nt being an abbreviation of nucleotide and representing a unit of the number of bases, 1nt having 2-bit information capacity, K=4200/(40*2)=53 (round up to an integer).

K=53, that is, the first data may be divided into 53 packet sub-data, and the length of generation times of the random generator must be greater than 53, the capacity of the random generator being T=3 nt. Since the 3 nt information storage capacity is 4³t(1 nt possesses a possibility of 4 base expressions, therefore, the information capacity is 1 nt is 4), it is also understood as 2⁶ (1 nt corresponds to 2 bits, and 1 bit corresponds to 0/1 states, therefore, there are 2 states of 3 (nt)*2 (bit)=6th-power information capacity).

By controlling the cycle number of the random generator, a plurality of random numbers may be output according to the input preset primer. Each random number is configured to select a portion of packet sub-data from K packet sub-data, and perform XOR operation on the selected portion of packet sub-data to obtain one data information DATAᵢ, i being the cycle number controlled, and 1≤i≤4^{T}.

Data information DATAᵢ is spliced with the preset primer and the generation times capacity of the random generator to obtain a DNA molecular chain, and 4^{T} DNA molecular chains are synthesized to obtain target storage data.

It can be seen that, in the process of coding the first data to obtain a DNA molecular chain, a random generator is added to greatly simplify the coding process and implement efficient and accurate coding on the first data. A primer of a DNA molecular chain is configured as a seed of a random generator to maximize the function of the primer; a preset ratio of the content of guanine and cytosine in the prefix of a molecular chain synthesized by each DNA to the total content of guanine, cytosine, adenine and thymine contained in the primer enables sequencing with high accuracy when coding data needs to be read in advance.

Further, as an optional implementation, block S2 includes blocks S21-S22:

at S21, a data length S and a packet length L of the first data are determined;

at S22, K packet sub-data is obtained according to the data length S and the packet length L.

Specifically, for example, the data length of the first data is S=4200 bit, the packet length is N=40 nt, the packet number K may be determined as:

$$K = ceil ( \frac{S}{N} ) = ceil \frac{4200 bit}{40 ⋆ 2 bit = 53}$$

ceil (.) being a round up integer function.

Further, as an optional implementation, block S3 is specifically:

controlling cycle number j, outputting a random integer in a range [0, 2K] according to the input preset primer by the random generator, and converting the random integer into a random number sequence DATA) in a binary form;

1≤j≤4^{T}.

Specifically, the preset primer is converted to a decimal integer as a seed into a random generator. The random generator outputs a decimal random integer in a range of [0, 2^{K}] according to the input primer, and converts the decimal random integer into a random number sequence in a binary form, and the high bit of the random number sequence is zeroed, so that the bit number of the random number sequence is K, and the binary is a degree distribution sequence of a random number sequence fountain code.

The cycle number j may be controlled by controlling a generation times capacity of a random generator to output 4^{K} random number sequences, 1≤j≤4^{K}.

Further, as an optional implementation, each random number sequence includes K random bits. Block S4 includes blocks S41-S42;

at S41, when it is judged that the value of the mth random bit of the ith random number sequence is 1, the packet sub-data corresponding to m random bits is selected, m being an integer and 1≤m≤K;

at S42, XOR operation is performed on the selected packet sub-data to obtain the data information DATAi.

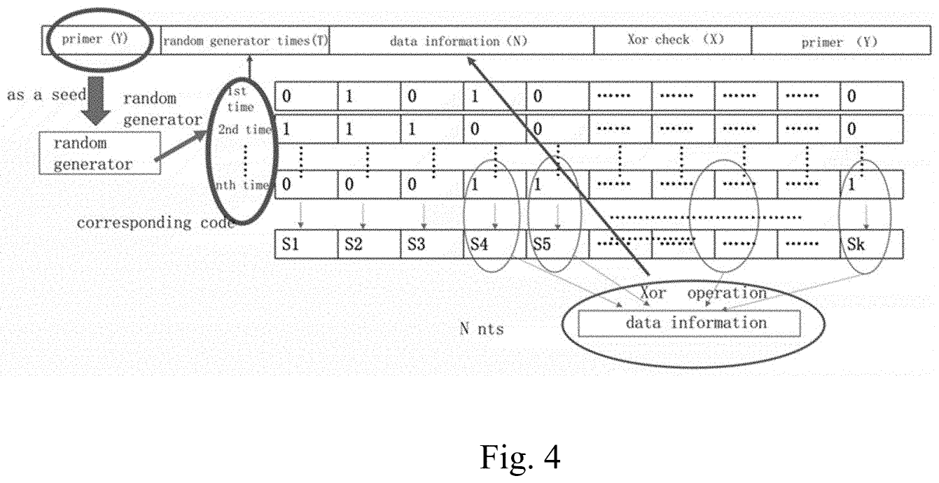

Specifically, referring to FIG. 4, each random number sequence is a random number sequence in a K-bit binary form, and each random bit of a random number sequence is judged; when it is determined that the number of the current random bit is 1, the packet sub-data corresponding to the random bit is selected, and XOR operation is performed on the selected plurality of packet sub-data to obtain data information corresponding to the current random number sequence.

According to the above way, by controlling the cycle number of the random number sequence, 4^{T} random number sequences correspond to 4^{T} data information. The preset primer, the generation times capacity of the random generator and the data information are assembled to form a set of fountain code drop data, that is, a DNA molecular chain.

Further, as an optional implementation, the storage method further includes randomization of a DNA molecular chain at S6. Block S6 includes S61-S62:

at S61, a preset primer is input into a random generator to obtain a random integer sequence;

at S62, the random integer sequence is converted into a binary sequence or a corresponding base sequence, a degree distribution sequence is generated under the guidance of the generation times of the random generator, and data information is guided to perform XOR operation.

Specifically, to ensure sufficient clutter of the finally generated target storage data, randomization is performed again on the basis of the DNA molecular chains generated in the previous block (that is, fountain code drop data), and the preset primer is converted to a decimal integer as a seed into a random generator to generate a random integer in a range of [0, 4^{T+N}] and the random integer is converted into a corresponding base sequence (or a corresponding binary sequence), and performs XOR operation with the random generation times capacity and the data information, to randomize the stored information.

Due to homopolymer imbalance or GC content imbalance in DNA storage, an unpredictable error may occur in the DNA sequence generation, PCR amplification and sequencing phases, so that when the DNA chain is synthesized, the homopolymer should be judged, and the situation that continuous 4 bases are the same base is discarded. Then, homopolymer and GC content are detected on the full chain. If not satisfy the requirement (4 bases are required not the same bases), the chain is deleted.

At last, DNA sequence synthesis is performed on the screened DNA molecular chains to obtain and store target storage data.

In addition, the disclosure further provides a decoding method applied to the target storage data obtained by the method for data storage. The method includes:

the target storage data is decoded.

The specific decoding process is as follow:

When data coding and storage are performed, preset primer information of DNA storage data and a data length for target storage data are known in advance. Meanwhile, a DNA sequence of the primer is also known. PCR amplification is performed according to primer information, and data is sequenced after amplification.

Block 1: the preset primer is converted to a decimal integer as a seed of a random generator into a random generator to generate a random number in a range of [0, 4^{T+N}] and the random number is converted to a corresponding base and performs XOR operation with a sequence in the DNA chain (target storage data) in addition to a base sequence of the preset primer to recover the original data.

Block 2: the preset primer is converted to a decimal integer as a seed of a random generator into a random generator according to the recovered data, and according to times information generated by the random generator, an integer in a range of [0, 2^{K}] is generated, and converted to a random number sequence in a K-bit binary form to record a next binary sequence D₁ and a data sequence DATA₁. Continue extracting a sequencing sequence until K different sequences are extracted, and K binary sequences D1, D2 . . . DK, and data sequences DATA1, DATA2 . . . DATAK are recorded.

Block 3: K K-bit sequence D is constitutes a K-order matrix D.

Block 4: a matrix solution is performed by a Gaussian elimination method. The K-order matrix D (represented by D₁, D₂ . . . D_{K}) and the K-row, 1-column DATA matrix (represented by DATA1, DATA2 . . . DATA_{K}) construct an augmented matrix. Then, judging along a diagonal of a matrix (i from 0−K), if D[i][i]=1, all the data of the ith row is XORed with all the data of the jth row. If D[i][i]=0, look down along the column; if D[j][i]=1, swap two rows and then look down; if another D[j][i]=1, the ith row is XORed with the jth row to ensure that an upper triangular matrix is constructed, and the area below the diagonal of the matrix is all 0.

Block 5: reverse operation is performed according to the previous block to eliminate all 1 above a diagonal to 0, further to obtain unique S1 . . . Sk, and a coding process is performed on DATA1 . . . DATA_{K}.

Then, refer to a system for data storage provided in embodiments of the disclosure with reference with the drawing.

FIG. 2 is a diagram of a structure of a system for data storage in one embodiment of the disclosure.

The system specifically includes:

a data acquiring module 201, configured to acquire first data;

a packet module 202, configured to group the first data to obtain K packet sub-data, the K being a positive integer;

a random number sequence acquiring module 203, configured to input a preset primer into a random generator to obtain 4^{T} random number sequences, T being a generation times capacity of the random generator, and 4^{T}>K, a preset ratio of the content of guanine and cytosine in the preset primer prefix to the total content of guanine, cytosine, adenine and thymine contained in the preset primer;

a packet determining module 204, configured to determine the packet sub-data corresponding to the ith random number sequence, and perform exclusive or (XOR) operation on the determined packet sub-data to obtain data information DATAi, i being a natural number and 1≤i≤4^{T}, and obtain a DNA molecular chain according to the data information DATAi, the preset primer and the generation times capacity of the random generator;

a synthesis module 205, configured to perform DNA sequence synthesis on the plurality of DNA molecular chains to obtain target storage data.

Further, as an optional implementation, each of the random number sequences includes K random bits. The packet determining module 204 includes:

a judging unit 2041, configured to, when judging that the value of the mth random bit of the ith random number sequence is 1, select the packet sub-data corresponding to m random bits, wherein, m being an integer and 1≤m≤K;

an XOR operation unit 2042, configured to perform XOR operation on the selected packet sub-data to obtain the data information DATAi.

It can be seen that the contents of the above method embodiments are applied to the system embodiments. The functions embodied in the system embodiments are the same as the functions of the above method embodiments, and the beneficial effects achieved are the same as the beneficial effects achieved by the above method embodiments.

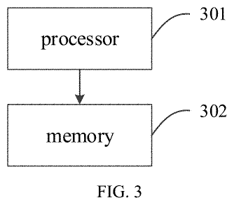

Referring to FIG. 3, the embodiments of the disclosure provide an apparatus for data storage. The apparatus includes:

at least one processor 301; and

at least one memory 302 configured to store at least one program;

when the at least one program is executed by the at least one processor 201, the at least one processor 201 is caused to implement the method for data storage.

The contents of the above method embodiments are applied to the apparatus embodiments. The functions embodied in the apparatus embodiments are the same as the functions of the above method embodiments, and the beneficial effects achieved are the same as the beneficial effects achieved by the above method embodiments.

In some optional embodiments, functions/operations referred to in block diagrams may occur not in accordance with sequence in the diagrams. For example, two blocks shown in succession may be executed substantially concurrently or sometimes may be executed in the reverse sequence, depending on functions/operations involved. In addition, the embodiments presented and described in the flowcharts of the present disclosure are provided by way of examples, and are intended to provide a more thorough understanding of the technology. The disclosed methods are not limited to operations and logic flows presented herein. Alternative embodiments are predictable. The sequence of various operations is changed and sub-operations described as a part of a larger operation are independently executed.

In addition, even though the disclosure is described in the context of a functional module, it should be understood that one or more of the above functions and/or features may be integrated in a single physical unit and/or software module, or one or more functions and/or features may be implemented in a separate physical device or software module, unless otherwise indicated. It may be further understood that the detailed discussion regarding the actual implementation of each module is not necessary for understanding the disclosure. More specifically, in consideration of attributes, functions and internal relationships of various functional modules in the apparatus disclosed herein, the actual implementation of the module may be understood by those skilled in the art. Accordingly, the disclosure as set forth in the claims may be implemented without undue tests by those skilled in the art. It may be further understood that specific concepts disclosed are illustrative only and are not intended to limit the scope of the disclosure defined by the appended claims and their entire scope of equivalents.

The above functions may be stored in a computer readable memory if it is implemented in the form of a software function unit and sold and used as an independent product On the basis of such an understanding, the technical solution of the present disclosure essentially or partly contributing to the related art, or part of the technical solution may be embodied in the form of a software product. The software product including several instructions is stored in a storage medium, so that a computer device (may be a personal computer, a server or a network device, etc.) executes all or part of blocks of various embodiments of the present disclosure. The medium includes a USB disk, a mobile hard disk, a read-only memory (ROM), a random access memory (RAM), a magnetic disk or an optical disk, and other media that may store program codes.

The logics and/or blocks represented in the flowchart or described in other ways herein, for example, may be considered as an ordered list of executable instructions configured to implement logic functions, which may be specifically implemented in any computer readable medium for use by instruction execution systems, apparatuses or devices (such as a computer-based system, a system including a processor, or other systems that may obtain and execute instructions from an instruction execution system, an apparatus or a device) or in combination with the instruction execution systems, apparatuses or devices. A “computer readable medium” in the specification may be an apparatus that may contain, store, communicate, propagate or transmit a program for use by instruction execution systems, apparatuses or devices or in combination with the instruction execution systems, apparatuses or devices.

A more specific example (a non-exhaustive list) of a computer readable medium includes the followings: an electronic connector (an electronic apparatus) with one or more cables, a portable computer disk box (a magnetic device), a random access memory (RAM), a read-only memory (ROM), an erasable programmable read-only memory (an EPROM or a flash memory), an optical fiber device, and a portable optical disk read-only memory (CDROM). In addition, a computer readable medium even may be paper or other suitable medium on which a program may be printed, since paper or other medium may be optically scanned, and then edited, interpreted or processed in other suitable ways if necessary to obtain a program electronically and store it in a computer memory.

It should be understood that all parts of the present disclosure may be implemented with a hardware, a software, a firmware and their combination. In the above implementation, multiple blocks or methods may be stored in a memory and implemented by a software or a firmware executed by a suitable instruction execution system. For example, if implemented with a hardware, they may be implemented by any of the following technologies or their combinations known in the art as in another implementation: a discrete logic circuit with logic gate circuits configured to achieve logic functions on data signals, a special integrated circuit with appropriate combined logic gate circuits, a programmable gate array (PGA), a field programmable gate array (FPGA), etc.

In the above descriptions, descriptions with reference to terms “one implementation/embodiment”, “another implementation/embodiment” or “some implementations/embodiments”, etc. mean specific features, structures, materials or characteristics described in combination with the implementation or example are included in at least one implementation or example of the present disclosure. The schematic representations of the above terms do not have to be the same implementation or example. Moreover, specific features, structures, materials or characteristics described may be combined in any one or more implementations or examples in a suitable manner.

Even though implementations of the disclosure have been illustrated and described, it may be understood by those skilled in the art that various changes, modifications, substitutions and alterations may be made for these implementations without departing from the principles and spirit of the disclosure, and the scope of the disclosure is defined by claims and their equivalents.

Although the preferred embodiments have been described in detail, the embodiments are not limited in the disclosure. Those skilled in the art know that various equivalent modifications or substitutions may be made without departing from the spirit of the disclosure, and all of these equivalent modifications or substitutions are intended to be included within the scope defined by the claims of the present disclosure.

1. A method for data storage, comprising:
acquiring first data;
grouping the first data to obtain K packet sub-data, wherein, the K being a positive integer;
inputting a preset primer into a random generator to obtain 4^{T} random number sequences, wherein, T being a generation times capacity of the random generator, and 4^{T}>K, a preset ratio of the content of guanine and cytosine in the preset primer prefix to the total content of guanine, cytosine, adenine and thymine contained in the preset primer;
determining the packet sub-data corresponding to the ith random number sequence, and performing exclusive or (XOR) operation on the determined packet sub-data to obtain data information DATAᵢ, wherein, i being a natural number and 1≤i≤4T, and obtaining a DNA molecular chain according to the data information DATAᵢ, the preset primer and the generation times capacity of the random generator;
performing DNA sequence synthesis on the plurality of DNA molecular chains to obtain target storage data.

2. The method of claim 1, wherein, grouping the first data to obtain K packet sub-data, comprising:
determining a data length and a packet length of the first data;
obtaining K packet sub-data according to the data length and the packet length.

3. The method of claim 1, wherein, inputting a preset primer into a random generator to obtain 4^{T} random number sequences, specifically:
controlling cycle number j, outputting a random integer in a range [0, 2^{K}] according to the input preset primer by the random generator, and converting the random integer into a random number sequence DATAⱼ in a binary form;
wherein, 1≤i≤4^{T}.

4. The method of claim 1, wherein, each of the random number sequences comprising K random bits, determining the packet sub-data corresponding to the ith random number sequence, and performing XOR operation on the determined packet sub-data to obtain data information DATAᵢ, comprising:
when judging that the value of the m^{th} random bit of the i^{th} random number sequence is 1, selecting the packet sub-data corresponding to m random bits, wherein, m being an integer and 1≤m≤K;
performing XOR operation on the selected packet sub-data to obtain the data information DATAᵢ.

5. The method of claim 1, wherein, further comprising randomization of the DNA molecular chain, comprising:
inputting a preset primer into a random generator to obtain a random integer sequence;
converting the random integer sequence into a binary sequence or a corresponding base sequence, generating a degree distribution sequence under the guidance of the generation times of the random generator, and guiding the data information to perform XOR operation.

6. (canceled)

7. A system for data storage, comprising:
a data acquiring module, configured to acquire first data;
a packet module, configured to group the first data to obtain K packet sub-data, wherein, the K being a positive integer;
a random number sequence acquiring module, configured to input a preset primer into a random generator to obtain 4^{T} random number sequences, wherein, T being a generation times capacity of the random generator, and 4^{T}>K, a preset ratio of the content of guanine and cytosine in the preset primer prefix to the total content of guanine, cytosine, adenine and thymine contained in the preset primer;
a packet determining module, configured to determine the packet sub-data corresponding to the ith random number sequence, and perform exclusive or (XOR) operation on the determined packet sub-data to obtain data information DATAᵢ, wherein, i being a natural number and 1≤i≤4T, and obtain a DNA molecular chain according to the data information DATAᵢ, the preset primer and the generation times capacity of the random generator;
a synthesis module, configured to perform DNA sequence synthesis on the plurality of DNA molecular chains to obtain target storage data.

8. The system of claim 7, wherein, each of the random number sequences comprising K random bits, the packet determining module comprising:
a judging unit, configured to, when judging that the value of the m^{th} random bit of the i^{th} random number sequence is 1, select the packet sub-data corresponding to m random bits, wherein, m being an integer and 1≤m≤K;
an XOR operation unit, configured to perform XOR operation on the selected packet sub-data to obtain the data information DATAᵢ.

9. (canceled)

10. (canceled)
